(19) United States
(12) Patent Application Publication
(10) Pub. No.: US 2026/0165996 A1
(43) Pub. Date: Jun. 18, 2026

(54) METHODS AND COMPOSITIONS FOR TREATING SLEEP APNEA

(71) Applicant: APNIMED, INC. (DELAWARE), Cambridge, MA (US)

(72) Inventors: Lawrence G. Miller, Cambridge, MA (US); Ronald Farkas, Clarksville, MD (US)

(21) Appl. No.: 19/537,913

(22) Filed: Feb. 12, 2026

Related U.S. Application Data
(63) Continuation of application No. 19/254,231, filed on Jun. 30, 2025, now abandoned.
Application No. 19/254,231 is a continuation of application No. 18/985,496, filed on Dec. 18, 2024, now abandoned.
Application No. 18/985,496 is a continuation of application No. 17/289,832, now abandoned.
(60) Provisional application No. 62/753,420, filed on Oct. 31, 2018.

(51) Int. Cl. A61K 31/216; A61K 31/138; A61K 31/5375; A61K 45/06

(52) U.S. Cl. CPC A61K 31/216; A61K 31/138; A61K 31/5375; A61K 45/06

(57) ABSTRACT

In general, the invention relates to pharmaceutical compositions comprising 4-hydroxyatomoxetine used with a muscarinic receptor antagonist, and methods of treating sleep apnea comprising administering a muscarinic receptor antagonist and 4-hydroxyatomoxetine. The 4-hydroxyatomoxetine and muscarinic receptor antagonist are disposed in a pharmaceutically acceptable carrier.

### CROSS REFERENCE TO RELATED APPLICATION

This application claims priority under 35 U.S.C. § 119 (e) to U.S. Provisional Application 62/753,420 filed on Oct. 31, 2018. The disclosure of this application is considered part of the disclosure of this application and is hereby incorporated by reference in its entirety.

### TECHNICAL FIELD

The present invention provides pharmaceutical compositions comprising 4-hydroxyatomoxetine and a muscarinic receptor antagonist, and methods of treating sleep apnea including administering 4-hydroxyatomoxetine and a muscarinic receptor antagonist.

### BACKGROUND

Obstructive Sleep Apnea (OSA) is a common disorder caused by collapse of the pharyngeal airway during sleep. OSA can have serious health consequences.

### SUMMARY

One aspect of the present invention provides a method of treating a subject having a condition associated with pharyngeal airway collapse, the method comprising administering to a subject in need thereof an effective amount of (i) 4-hydroxyatomoxetine; and (ii) a muscarinic receptor antagonist.

Embodiments of this aspect of the invention may include one or more of the following optional features. In some embodiments, the muscarinic receptor antagonist comprises oxybutynin. In some embodiments, the oxybutynin is a substantially pure (R)-oxybutynin. In some embodiments, the oxybutynin is a racemic mixture of (R)-oxybutynin and(S)-oxybutynin. In some embodiments, an enantiomeric excess of (R)-oxybutynin relative to(S)-oxybutynin is greater than about 25% (e.g., greater than about 50% or greater than about 75%). In some embodiments, the oxybutynin is in an immediate release formulation. In some embodiments, the oxybutynin is in an extended release formulation. In some embodiments, the oxybutynin is present in an amount of from about 2 to about 15 mg. For example, the oxybutynin may be in an immediate release formulation and may be present in an amount of from about 2.5 to about 10 mg. Or for example, the oxybutynin may be in an extended release formulation and may be present in an amount of from about 5 to about 15 mg. In some embodiments, the method further comprises administering an effective amount of a norepinephrine selective reuptake inhibitor (NSRI) selected from the group consisting of Amedalin, Atomoxetine, CP-39,332, Daledalin, Edivoxetine, Esreboxetine, Lortalamine, Nisoxetine, Reboxetine, Talopram, Talsupram, Tandamine, Viloxazine and combinations thereof. In some embodiments, the method further comprises administering an effective amount of a norepinephrine non-selective reuptake inhibitor (NNRI) selected from the group consisting of Amitriptiline, Amoxapine, Bupropion, Ciclazindol, Desipramine, Desvenlafaxine, Dexmethilphenidate, Diethylpropion, Doxepin, Duloxetine, Imipramine, Levomilnacipran, Manifaxine, Maprotiline, Methylphenidate, Milnacipran, Nefazodone, Nortriptyline, Phendimetrazine, Protryptyline, Radafaxine, Tapentadol, Teniloxazine, Venlafaxine, and combinations thereof. In some embodiments, the method further comprises administering an effective amount of Reboxetine. In some embodiments, the method further comprises administering an effective amount of a hypnotic selected from the group consisting of zolpidem, zopiclone, eszopiclone, trazodone, zaleplon, benzodiazepines, gabapentin, tiagabine, and xyrem. In some embodiments, the 4-hydroxyatomoxetine is administered at a dose of from about 20 to about 100 mg (e.g., about 25 to about 75 mg). In some embodiments, the condition associated with pharyngeal airway collapse is sleep apnea or simple snoring. In some embodiments, the condition associated with pharyngeal airway collapse is Obstructive Sleep Apnea (OSA). In some embodiments, the subject is in a non-fully conscious state (e.g., sleep). In some embodiments, the 4-hydroxyatomoxetine and muscarinic receptor antagonist are administered in a single composition. In some embodiments, the single composition is an oral administration form (e.g., a syrup, pill, tablet, troche, capsule, or patch).

Another aspect of the present invention provides a pharmaceutical composition comprising: 4-hydroxyatomoxetine; and a muscarinic receptor antagonist disposed in a pharmaceutically acceptable carrier.

Embodiments of this aspect of the invention may include one or more of the following optional features. In some embodiments, the muscarinic receptor antagonist comprises oxybutynin. In some embodiments, the oxybutynin is a substantially pure (R)-oxybutynin. In some embodiments, the oxybutynin is a racemic mixture of (R)-oxybutynin and(S)-oxybutynin. In some embodiments, an enantiomeric excess of (R)-oxybutynin relative to(S)-oxybutynin is greater than about 25% (e.g., greater than about 50% or greater than about 75%). In some embodiments, the oxybutynin is in an immediate release formulation. In some embodiments, the oxybutynin is in an extended release formulation. In some embodiments, the oxybutynin is present in an amount of from about 2 to about 15 mg. For example, the oxybutynin may be in an immediate release formulation and may be present in an amount of from about 2.5 to about 10 mg. Or for example, the oxybutynin may be in an extended release formulation and may be present in an amount of from about 5 to about 15 mg. In some embodiments, the 4-hydroxyatomoxetine and muscarinic receptor antagonist are disposed together in a pharmaceutically acceptable carrier to form a single dosage agent. In some embodiments, the 4-hydroxyatomoxetine and muscarinic receptor antagonist are separately disposed in pharmaceutically acceptable carriers to form a separate 4-hydroxyatomoxetine dosing agent and a muscarinic receptor antagonist dosing agent. In some embodiments, the pharmaceutical composition further comprises a norepinephrine selective reuptake inhibitor (NSRI) selected from the group consisting of Amedalin, Atomoxetine, CP-39,332, Daledalin, Edivoxetine, Esreboxetine, Lortalamine, Nisoxetine, Reboxetine, Talopram, Talsupram, Tandamine, Viloxazine, and combinations thereof. In some embodiments, the pharmaceutical composition further comprises a norepinephrine non-selective reuptake inhibitor (NNRI) selected from the group consisting of Amitriptiline, Amoxapine, Bupropion, Ciclazindol, Desipramine, Desvenlafaxine, Dexmethilphenidate, Diethylpropion, Doxepin, Duloxetine, Imipramine, Levomilnacipran, Manifaxine, Maprotiline, Methylphenidate, Milnacipran, Nefazodone, Nortriptyline, Phendimetrazine, Protryptyline, Radafaxine, Tapentadol, Teniloxazine, Venlafaxine, and combinations thereof. In some embodiments, the pharmaceutical composition further comprises Reboxetine. In some embodiments, the pharmaceutical composition further comprises a hypnotic selected from the group consisting of zolpidem, zopiclone, eszopiclone, trazodone, zaleplon, benzodiazepines, gabapentin, tiagabine, and xyrem. In some embodiments, the 4-hydroxyatomoxetine is present in an amount of from about 20 to about 100 mg (e.g., about 25 to about 75 mg). In some embodiments, the composition is for use in treating a subject having a condition associated with pharyngeal airway collapse. In some embodiments, the condition associated with pharyngeal airway collapse is sleep apnea or simple snoring. In some embodiments, the condition associated with pharyngeal airway collapse is Obstructive Sleep Apnea (OSA). In some embodiments, the subject is in a non-fully conscious state (e.g., sleep).

Another aspect of the invention provides 4-hydroxyatomoxetine and a muscarinic receptor antagonist for use in treating a subject having a condition associated with pharyngeal airway collapse.

Still another aspect of the invention provides a kit comprising 4-hydroxyatomoxetine and a muscarinic receptor antagonist. In some embodiments, the kit is for use in treating a subject having a condition associated with pharyngeal airway collapse.

Another aspect of the invention provides a method of treating a subject having a condition associated with pharyngeal airway collapse, the method comprising: administering to a subject in need thereof an effective amount of a muscarinic receptor antagonist; and modulating a norepinephrine transporter (NET) of the subject in vivo with an effective amount of 4-hydroxyatomoxetine.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

The following FIGURES are provided by way of example and are not intended to limit the scope of the claimed invention.

FIG. 1. Graphic illustration of an obstructive apnea. The top channel shows the electroencephalogram (EEG) pattern of sleep. The next channel represents airflow. The next three channels show ventilator effort by movements of the rib cage and abdomen and changes in esophageal pressure, all of which reflect contraction of respiratory muscles. The last channel indicates oxyhemoglobin saturation.

### DETAILED DESCRIPTION

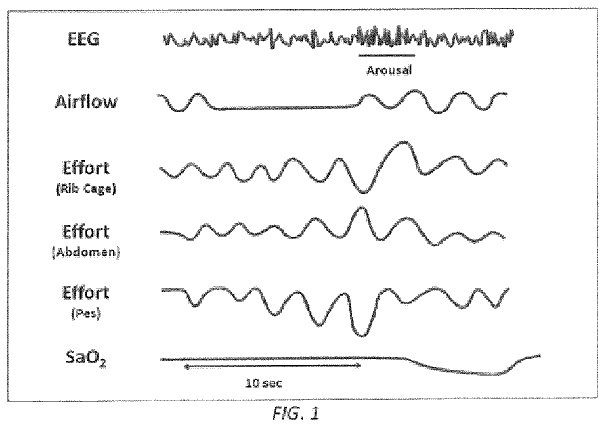

In humans, the pharyngeal airway region has no bone or cartilage support, and it is held open by muscles. When these muscles relax during sleep, the pharynx can collapse resulting in cessation of airflow. As shown in FIG. 1, ventilatory effort continues and increases in an attempt to overcome the obstruction, shown by an increase in esophageal pressure change. Rib cage and abdominal movements are in the opposite direction as a result of the diaphragm contracting against an occluded airway, forcing the abdominal wall to distend out and the chest wall to cave inward.

Increasing efforts to breathe lead to an arousal from sleep, visualizable on an EEG (FIG. 1), and result in opening of the airway and a resumption of normal breathing. The lack of airflow during the apnea also causes hypoxia, shown by a drop in oxyhemoglobin saturation (FIG. 1). Severity is generally measured using the apnea-hypopnea index (AHI), which is the combined average number of apneas (cessation of breathing for at least ten seconds) and hypopneas (reduced airflow and oxygen saturation) that occur per hour of sleep (Ruehland et al., The new AASM criteria for scoring hypopneas: Impact on the apnea hypopnea index. SLEEP 2009; 32 (2): 150-157).

When a stringent definition of OSA is used (an AHI of >15 events per hour or AHI >5 events per hour with daytime sleepiness), the estimated prevalence is approximately 15 percent in males and 5 percent in females. An estimated 30 million individuals in the United States have OSA, of which approximately 6 million have been diagnosed. The prevalence of OSA in the United States appears to be increasing due to aging and increasing rates of obesity. OSA is associated with major comorbidities and economic costs, including: hypertension, diabetes, cardiovascular disease, motor vehicle accidents, workplace accidents, and fatigue/lost productivity. (Young et al., WMJ 2009; 108:246; Peppard et al., Am J Epidemiol 2013; 177:1006.)

The present leading treatment is continuous positive airway pressure (CPAP). CPAP is effective in virtually all patients, and approximately 85% of diagnosed patients are treated, but compliance is low. Patients find CPAP uncomfortable and often intolerable; at least 30% of patients (up to 80%) are regularly non-adherent and thus untreated (Weaver, Proc Am Thorac Soc. 2008 Feb. 15; 5 (2): 173-178). Other treatment modalities with variable rates of success include oral appliances (10%) and surgery (5%), but neither is likely to be effective across the general population. No pharmacologic treatments have been shown to be effective to date.

The search for medicines to activate pharyngeal muscles in sleeping humans has been discouraging; agents such as serotonin reuptake inhibitors, tricyclic antidepressants, and sedatives have all been tested in humans and shown to be ineffective at reducing OSA severity. See, e.g., Proia and Hudgel, Chest. 1991 August; 100 (2): 416-21; Brownell et al., N Engl J Med 1982, 307:1037-1042; Sangal et al., Sleep Med. 2008 Jul; 9 (5): 506-10. Epub 2007 Sep. 27; Marshall et al. p. 2008 Jun; 31 (6): 824-31; Eckert et al., Clin Sci (Lond). 2011 June; 120 (12); 505-14; Taranto-Montemurro et al., Sleep. 2017 Feb. 1; 40 (2).

### Methods of Treatment

The methods described herein include methods for the treatment of disorders associated with pharyngeal airway muscle collapse during sleep. In some embodiments, the disorder is Obstructive Sleep Apnea (OSA) or Simple Snoring. Generally, the methods include administering a therapeutically effective amount of a 4-hydroxyatomoxetine norepinephrine reuptake inhibitor and an oxybutynin muscarinic receptor antagonist as described herein, to a subject who is in need of, or who has been determined to be in need of, such treatment.

As used in this context, to “treat” means to ameliorate at least one symptom of the disorder associated with pharyngeal airway collapse. Often, pharyngeal airway collapse during sleep results in snoring and/or an interruption in breathing (apnea or hypopnea), arousal from sleep, and reduced oxygenation (hypoxemia); thus, a treatment can result in a reduction in snoring, apneas/hypopneas, sleep fragmentation, and hypoxemia. Administration of a therapeutically effective amount of a compound described herein for the treatment of a subject with OSA will result in decreased AHI.

An effective amount can be administered in one or more administrations, applications or dosages. The compositions can be administered from one or more times per day to one or more times per week; including once every other day. In some embodiments, the compositions are administered daily. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds (i.e., 4-hydroxyatomoxetine and oxybutynin, in a single composition or in separate compositions) can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

In some embodiments, the methods include administering a dose of 20-100 mg 4-hydroxyatomoxetine (or a dose equivalent thereof in combination with another NRI) and a dose of 2-15 mg oxybutynin (racemic mixture). In some embodiments, the methods include administering 75 mg 4-hydroxyatomoxetine/6 mg oxybutynin; 50 mg 4-hydroxyatomoxetine/4 mg oxybutynin; or 25 mg 4-hydroxyatomoxetine/3 mg oxybutynin, e.g., 15-60, e.g., 15-25, 20-30, or 20-45 minutes before sleep time.

In other embodiments, the methods include administering a dose of 20-100 mg 4-hydroxyatomoxetine (or a dose equivalent thereof in combination with another NRI) and a dose of 2-15 mg (R)-oxybutynin. In some embodiments, the methods include administering 75 mg 4-hydroxyatomoxetine/6 mg (R)-oxybutynin; 50 mg 4-hydroxyatomoxetine/4 mg (R)-oxybutynin; or 25 mg 4-hydroxyatomoxetine/3 mg (R)-oxybutynin, e.g., 15-60, e.g., 15-25, 20-30, or 20-45 minutes before sleep time.

### Pharmaceutical Compositions and Methods of Administration

The methods described herein include the use of pharmaceutical compositions comprising 4-hydroxyatomoxetine and a muscarinic receptor antagonist comprising oxybutynin (e.g., N-desethyloxybutynin) as active ingredients. The 4-hydroxyatomoxetine norepinephrine reuptake inhibitor and oxybutynin muscarinic receptor antagonist can be administered in a single composition or in separate compositions.

In some embodiments, Atomoxetine can be metabolized by cytochrome 2D6 to form 4-hydroxyatomoxetine. The 4-hydroxyatomoxetine metabolite demonstrates similar reactivity and physiological responses as the parent Atomoxetine. The 4-hydroxyatomoxetine may then be subsequently glucuronidated to form 4-hydroxyatomoxitine-O-glucuronide and can be eliminated from the body in urine. Scheme 1 below illustrates an exemplary metabolic pathway of Atomoxetine in humans.

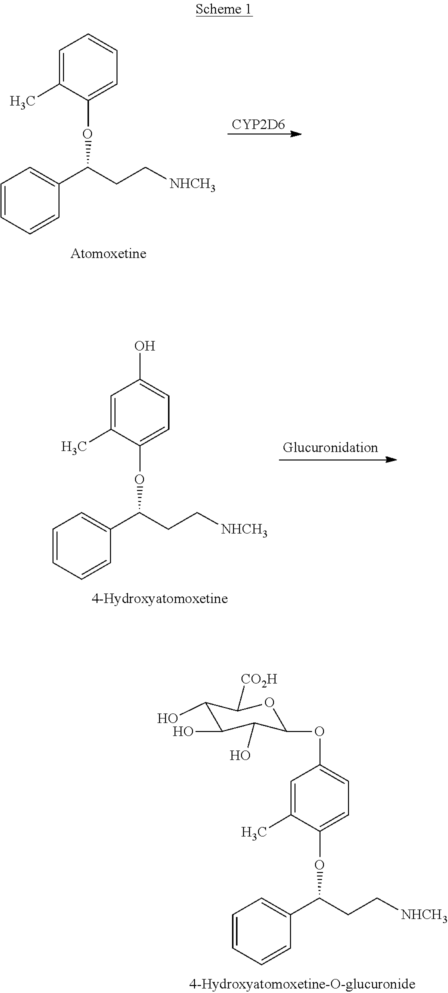 [SMILES: CNCC[C@@H](Oc1ccccc1C)c1ccccc1 ; "CYP2D6" ; CNCC[C@@H](Oc1ccc(O)cc1C)c1ccccc1 ; "Glucuronidation" ; CNCC[C@@H](Oc1ccc(OC2OC(C(=O)O)C(O)C(O)C2O)cc1C)c1ccccc1]

Administering 4-hydroxyatomoxetine to a patient can provide several advantages. For example, since 4-hydroxyatomoxetine is not typically subjected to further metabolism by the cytochrome 2D6 enzyme, the variability of additional atomoxetine metabolites is reduced so the concentration and delivery of 4-hydroxyatomoxetine can be expected to be more uniform across the patient population. The improved delivery and blood concentration of 4-hydroxyatomoxetine across patient populations can lead to improved dosing regimens that provide patients with better therapeutic responses to OSA and pharyngeal airway collapse.

In some embodiments, as provided in Scheme 1, the 4-hydroxyatomoxetine used to treat a subject have a condition associated with pharyngeal airway collapse is substantially enantiomerically pure (R)-4-hydroxyatomoxetine or (R)—N-Methyl-3-phenyl-3-(o-tolyloxy) propan-1-amine. In other embodiments, the 4-hydroxyatomoxetine may include a mixture of (R)-4-hydroxyatomoxetine and(S)-4-hydroxyatomoxetine. A composition comprising substantially enantiomerically pure (R)-4-hydroxyatomoxetine, as described herein, may have an enantiomeric excess of the substantially enantiomerically pure (R)-4-hydroxyatomoxetine of >80%, ≥90%, ≥95%, >98%, ≥99%, ≥99.5%, ≥99.8% or ≥99.9%.

Exemplary additional norepinephrine reuptake inhibitors (NRIs) include the selective NRIs Amedalin (UK-3540-1), Atomoxetine (Strattera), CP-39,332, Daledalin (UK-3557-15), Edivoxetine (LY-2216684), Esreboxetine, Lortalamine (LM-1404), Nisoxetine (LY-94,939), Reboxetine (Edronax, Vestra), Talopram (Lu 3-010), Talsupram (Lu 5-005), Tandamine (AY-23,946), Viloxazine (Vivalan), and combinations thereof.

In some embodiments, the norepinephrine reuptake inhibitors (NRIs) further comprise non-selective norepinephrine reuptake inhibitors (NNRIs) including, for example, Amitriptiline, Amoxapine, Bupropion, Ciclazindol, Desipramine, Desvenlafaxine, Dexmethilphenidate, Diethylpropion, Doxepin, Duloxetine, Imipramine, Levomilnacipran, Manifaxine (GW-320,659), Maprotiline, Methylphenidate, Milnacipran, Nefazodone, Nortriptyline, Phendimetrazine, Phenmetrazine, Protryptyline, Radafaxine (GW-353,162), Tapentadol (Nucynta), Teniloxazine (Lucelan, Metatone), Venlafaxine, and combinations thereof.

In some embodiments, the norepinephrine reuptake inhibitor is 4-hydroxyatomoxetine.

Oxybutynin is an antimuscarinic drug and a muscarinic receptor antagonist. In some embodiments, the oxybutynin is a racemic mixture of (R)-oxybutynin and(S)-oxybutynin where the enantiomers are present in about equal stoichiometric amounts. A composition comprising a mixture of oxybutynin enantiomers, as described herein, may further comprise an enantiomeric excess of (R)-oxybutynin relative to its enantiomeric pair (i.e., (S)-oxybutynin). The enantiomeric excess of (R)-oxybutynin in these mixtures may be ≥10%, ≥20%, ≥25%, ≥30%, ≥40%, ≥50%, ≥60%, ≥70%, ≥75%, ≥80%, or ≥90%.

In some embodiments, the muscarinic receptor antagonist is a substantially enantiomerically pure (R)-oxybutynin. A composition comprising substantially enantiomerically pure (R)-oxybutynin, as described herein, may have an enantiomeric excess of the substantially enantiomerically pure (R)-oxybutynin of ≥80%, ≥90%, ≥95%, ≥98%, ≥99%, ≥99.5%, ≥99.8% or ≥99.9%.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., hypnotics including zolpidem, zopiclone, eszopiclone, trazodone, zaleplon, benzodiazepines, gabapentin, tiagabine, and xyrem. In some embodiments, patients having OSA have a low arousal threshold, which can be exacerbated by atomoxetine and/or 4-hydroxyatomextine. In such embodiments where patients have a low arousal threshold caused or worsened by the use of atomoxetine and/or 4-hydroxyatomextine, a hypnotic can be used as a supplementary active compound to increase the arousal threshold of the patient having OSA, pharyngeal airway collapse, or a combination thereof. In some embodiments, the arousal threshold of a patient can be measured by polysomnography (PSG). In some embodiments, the pharmaceutical composition comprises 4-hydroxyatomextine, a muscarinic receptor antagonist, and a hypnotic. In some embodiments, the method of treating a subject having a condition associated with pharyngeal airway collapse comprises administering to a subject in need thereof an effective amount of (i) 4-hydroxyatomoxetine; (ii) a muscarinic receptor antagonist, and (iii) a hypnotic.

In some embodiments, the methods include administering a dose of 20-100 mg 4-hydroxyatomoxetine, a dose of 2-15 mg oxybutynin (i.e., muscarinic receptor antagonist), and a dose of 0.5-15 mg zolpidem (or a dose equivalent thereof of another hypnotic). In some embodiments, the methods include administering 75 mg 4-hydroxyatomoxetine/6 mg oxybutynin/10 mg zolpidem; 75 mg 4-hydroxyatomoxetine/5 mg oxybutynin/10 mg zolpidem; 75 mg 4-hydroxyatomoxetine/4.5 mg oxybutynin/5 mg zolpidem; 50 mg 4-hydroxyatomoxetine/4 mg oxybutynin/3.5 mg zolpidem; or 25 mg 4-hydroxyatomoxetine/3 mg oxybutynin/1.75 mg zolpidem, e.g., 15-60, 15-25, 20-30, or 20-45 minutes before sleep time. In some embodiments, the hypnotic is present in an amount of from about 0.5 to about 15 mg, from about 0.5 to about 10 mg, from about 0.5 to about 5 mg, from about 0.5 to about 3.5 mg, or from about 0.5 to about 1.75 mg. In some embodiments, the 4-hydroxyatomoxetine, muscarinic receptor antagonist (e.g., oxybutynin), and hypnotic are administered in a single composition, for example, an oral administration in a syrup, pill, tablet, capsule, or patch form.

Pharmaceutical compositions are typically formulated to be compatible with its intended route of administration. Examples of routes of administration include systemic oral or transdermal administration.

Methods of formulating suitable pharmaceutical compositions using pharmaceutically acceptable carriers are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, oral compositions generally include an inert diluent or an edible carrier. For the purpose of oral therapeutic administration, the active compound(s) can be incorporated with excipients and used in the form of pills, tablets, troches, or capsules, e.g., gelatin capsules. Oral compositions can also be prepared using a fluid carrier. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In some embodiments, the 4-hydroxyatomoxetine and muscarinic receptor antagonist are disposed together in one or more pharmaceutically acceptable carriers to form a single dosage agent. The single dosage agent comprises 4-hydroxyatomoxetine, muscarinic receptor antagonist, and the pharmaceutically acceptable carrier. In other embodiments, the 4-hydroxyatomoxetine and muscarinic receptor antagonist are separately disposed in one or more pharmaceutically acceptable carriers to form a separate 4-hydroxyatomoxetine dosing agent and a muscarinic receptor antagonist dosing agent. The separate 4-hydroxyatomoxetine and muscarinic receptor antagonist dosing agents may be used in any combination to provide treatment to a subject having a condition associated with pharyngeal airway collapse, sleep apnea, or a combination thereof.

Systemic administration of one or both of the compounds as described herein (i.e., one or both of the 4-hydroxyatomoxetine norepinephrine reuptake inhibitor and the oxybutynin muscarinic receptor antagonist) can also be by transdermal means, e.g., using a patch, gel, or lotion, to be applied to the skin. For transdermal administration, penetrants appropriate to the permeation of the epidermal barrier can be used in the formulation. Such penetrants are generally known in the art. For example, for transdermal administration, the active compounds can formulated into ointments, salves, gels, or creams as generally known in the art. The gel and/or lotion can be provided in individual sachets, or via a metered-dose pump that is applied daily; see, e.g., Cohn et al., Ther Adv Urol. 2016 April; 8 (2): 83-90.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

In some embodiments, a method of treating a subject having a condition associated with pharyngeal airway collapse is provided. The method comprises administering to the subject in need thereof an effective amount of a muscarinic receptor antagonist, and modulating a norepinephrine transporter (NET) of the subject in vivo with an effective amount of 4-hydroxyatomoxetine. 4-Hydroxyatomoxetine is a norepinephrine (noradrenaline) reuptake inhibitor that can inhibit presynaptic norepinephrine transporters, preventing the reuptake of norepinephrine throughout the brain along with inhibiting the reuptake of dopamine in specific brain regions such as the prefrontal cortex.

The pharmaceutical compositions can be included in a container, pack, or dispenser together with instructions for administration or use in a method described herein.

### EXAMPLES

The invention is further described in the following examples, which do not limit the scope of the invention described in the claims.

### Example 1. Pilot Study Using 4-Hydroxyatomoxetine and Racemic Oxybutynin

In healthy human individuals, the effect of the selective noradrenergic reuptake inhibitor, 4-hydroxyatomoxetine 80 mg, in combination with the antimuscarinic drug oxybutynin 5 mg (racemic mixture) on genioglossus muscle activity is measured in a pilot study.

A first group of the patients is given the combination of 4-hydroxyatomoxetine 80 mg and oxybutynin 5 mg. A second group of patients is given placebo. Genioglossus muscle activity (EMG_{GG}, quantified as a percentage of maximum) is measured during quiet wakefulness. Each peak EMG_{GG} of a single breath is measured and is plotted against the corresponding epiglottic pressure. In addition, EMG_{GG} is measured during stable NREM sleep.

It is expected that there will be a variable but clear reduction in EMG_{GG} activity during sleep on the placebo night and that, in contrast, when patients are administered 4-hydroxyatomoxetine+oxybutynin, the sleep-related reduction in pharyngeal muscle activity will be partially or completely prevented.

It is expected that, compared to placebo, the tested drugs will yield a much higher EMG_{GG} activity during NREM sleep. It is also expected that the drugs will be effective during REM sleep for those subjects exhibiting REM sleep when administered the tested drugs.

### Example 2. Crossover Study Using 4-Hydroxyatomoxetine and Racemic Oxybutynin

A placebo-controlled, double-blinded, randomized, crossover trial in obstructive sleep apnea (OSA) human patients is performed. Participants receive treatment (4-hydroxyatomoxetine 80 mg+oxybutynin 5 mg) or placebo in randomized order 30 minutes before sleep. The combination of 4-hydroxyatomoxetine and oxybutynin is expected to reduce the apnea hypopnea index and all patients are expected to experience an improvement in OSA severity. Additional benefits expected are increased genioglossus muscle responsiveness to an increase in ventilatory drive, improved upper airway muscle activity, improved ventilation, increased oxygen levels (SaO₂), increased total sleep time and improved sleep efficiency.

### Example 3. Pilot Study Using 4-Hydroxyatomoxetine and (R)-Oxybutynin

In healthy human individuals, the effect of the selective noradrenergic reuptake inhibitor, 4-hydroxyatomoxetine 80 mg, in combination with the antimuscarinic drug (R)-oxybutynin 5 mg on genioglossus muscle activity is measured in a pilot study.

A first group of the patients is given the combination of 4-hydroxyatomoxetine 80 mg and (R)-oxybutynin 5 mg. A second group of patients is given placebo. Genioglossus muscle activity (EMG_{GG}, quantified as a percentage of maximum) is measured during quiet wakefulness. Each peak EMG_{GG} of a single breath is measured and is plotted against the corresponding epiglottic pressure. In addition, EMG_{GG} is measured during stable NREM sleep.

It is expected that there will be a variable but clear reduction in EMG_{GG} activity during sleep on the placebo night and that, in contrast, when patients are administered 4-hydroxyatomoxetine+(R)-oxybutynin, the sleep-related reduction in pharyngeal muscle activity will be partially or completely prevented.

It is expected that, compared to placebo, the tested drugs will yield a much higher EMG_{GG} activity during NREM sleep. It is also expected that the drugs will be effective during REM sleep for those subjects exhibiting REM sleep when administered the tested drugs.

### Example 4. Crossover Study Using 4-Hydroxyatomoxetine and (R)-Oxybutynin

A placebo-controlled, double-blinded, randomized, crossover trial in obstructive sleep apnea (OSA) human patients is performed. Participants receive treatment (4-hydroxyatomoxetine 80 mg+(R)-oxybutynin 5 mg) or placebo in randomized order 30 minutes before sleep. The combination of 4-hydroxyatomoxetine and (R)-oxybutynin is expected to reduce the apnea hypopnea index and all patients are expected to experience an improvement in OSA severity. Additional benefits expected are increased genioglossus muscle responsiveness to an increase in ventilatory drive, improved upper airway muscle activity, improved ventilation, increased oxygen levels (SaO₂), increased total sleep time and improved sleep efficiency.

### REFERENCES

1. Young T, Peppard P E, Gottlieb D J. Epidemiology of obstructive sleep apnea: a population health perspective. Am J Respir Crit Care Med 2002; 165:1217-39.
2. Engleman H M, Wild M R. Improving CPAP use by patients with the sleep apnea/hypopnea syndrome (SAHS). Sleep Med Rev 2003; 7:81-99.
3. Kribbs N B, Pack Al, Kline L R, et al. Objective measurement of patterns of nasal CPAP use by patients with obstructive sleep apnea. The American review of respiratory disease 1993; 147:887-95.
4. Chan E, Steenland H W, Liu H, Homer R L. Endogenous excitatory drive modulating respiratory muscle activity across sleep-wake states. American journal of respiratory and critical care medicine 2006; 174:1264-73.
5. Grace K P, Hughes S W, Homer R L. Identification of the mechanism mediating genioglossus muscle suppression in REM sleep. Am J Respir Crit Care Med 2013; 187:311-9.
6. Kubin L, Davies R O, Pack A I. Control of Upper Airway Motoneurons During REM Sleep. News Physiol Sci 1998; 13:91-7.
7. Sood S, Morrison J L, Liu H, Horner R L. Role of endogenous serotonin in modulating genioglossus muscle activity in awake and sleeping rats. American journal of respiratory and critical care medicine 2005; 172:1338-47.
8. Sood S, Raddatz E, Liu X, Liu H, Homer R L. Inhibition of serotonergic medullary raphe obscurus neurons suppresses genioglossus and diaphragm activities in anesthetized but not conscious rats. J Appl Physiol (1985) 2006; 100:1807-21.
9. Fenik V B, Davies R O, Kubin L. REM sleep-like atonia of hypoglossal (XII) motoneurons is caused by loss of noradrenergic and serotonergic inputs. Am J Respir Crit Care Med 2005; 172:1322-30.
10. Sood S, Liu X, Liu H, Horner R L. Genioglossus muscle activity and serotonergic modulation of hypoglossal motor output in obese Zucker rats. J Appl Physiol (1985) 2007; 102:2240-50.
11. Hanzel D A, Proia N G, Hudgel D W. Response of obstructive sleep apnea to fluoxetine and protriptyline. Chest 1991; 100:416-21.
12. Kraiczi H, Hedner J, Dahlof P, Ejnell H, Carlson J. Effect of serotonin uptake inhibition on breathing during sleep and daytime symptoms in obstructive sleep apnea. Sleep 1999; 22:61-7.
13. Berry R B, Yamaura E M, Gill K, Reist C. Acute effects of paroxetine on genioglossus activity in obstructive sleep apnea. Sleep 1999; 22:1087-92.
14. Lai Y Y, Kodama T, Siegel J M. Changes in monoamine release in the ventral horn and hypoglossal nucleus linked to pontine inhibition of muscle tone: an in vivo microdialysis study. J Neurosci 2001; 21:7384-91.
15. Grace K P, Hughes S W, Shahabi S, Horner R L. K+ channel modulation causes genioglossus inhibition in REM sleep and is a strategy for reactivation. Respir Physiol Neurobiol 2013; 188:277-88.
16. Eckert D J, White D P, Jordan A S, Malhotra A, Wellman A. Defining phenotypic causes of obstructive sleep apnea. Identification of novel therapeutic targets. Am J Respir Crit Care Med 2013; 188:996-1004.
17. Wellman A, Eckert D J, Jordan A S, Edwards B A, Passaglia C L, Jackson A C, Gautam S, Owens R L, Malhotra A, White D P. A method for measuring and modeling the physiological traits causing obstructive sleep apnea. J Appl Physiol 2011; 110:1627-1637.
18. Wellman A, Edwards B A, Sands S A, Owens R L, Nemati S, Butler J P, Passaglia C L, Jackson A C, Malhotra A, White D P. A simplified method for determining phenotypic traits in patients with obstructive sleep apnea. J Appl Physiol 2013.
19. Younes M. Contributions of upper airway mechanics and control mechanisms to severity of obstructive apnea. Am J Respir Crit Care Med 2003; 168:645-658.
20. Somers V K, Dyken M E, Clary M P, Abboud F M. Sympathetic neural mechanisms in obstructive sleep apnea. J Clin Invest 1995; 96:1897-1904.
21. Nieto F J, Young T B, Lind B K, Shahar E, Samet J M, Redline S, D'Agostino R B, Newman A B, Lebowitz M D, Pickering T G. Association of sleep-disordered breathing, sleep apnea, and hypertension in a large community-based study. Sleep heart health study. Jama 2000; 283:1829-1836.
22. Brooks D, Homer R L, Kozar L F, Render-Teixeira C L, Phillipson E A. Obstructive sleep apnea as a cause of systemic hypertension. Evidence from a canine model. J Clin Invest 1997; 99:106-109.
23. Peppard P E, Young T, Palta M, Skatrud J. Prospective study of the association between sleep-disordered breathing and hypertension. The New England journal of medicine 2000; 342:1378-1384.
24. Hung J, Whitford E G, Parsons R W, Hillman D R. Association of sleep apnea with myocardial infarction in men. Lancet 1990; 336:261-264.
25. Wessendorf T E, Teschler H, Wang Y M, Konietzko N, Thilmann A F. Sleep-disordered breathing among patients with first-ever stroke. J Neurol 2000; 247:41-47.
26. Hoffstein V. Blood pressure, snoring, obesity, and nocturnal hypoxaemia. Lancet 1994; 344:643-645.
27. Shahar E, Whitney C W, Redline S, Lee E T, Newman A B, Nieto F J, O'Connor G T, Boland L L, Schwartz J E, Samet J M. Sleep-disordered breathing and cardiovascular disease: Cross-sectional results of the sleep heart health study. Am J Respir Crit Care Med 2001; 163:19-25.
28. Redline S, Strauss M E, Adams N, Winters M, Roebuck T, Spry K, Rosenberg C, Adams K. Neuropsychological function in mild sleep-disordered breathing. Sleep 1997; 20:160-167.
29. Findley L I, Unverzagt M E, Suratt P M. Automobile accidents involving patients with obstructive sleep apnea. Am Rev Respir Dis 1988; 138:337-340.
30. Edwards B A, Sands S A, Eckert D J, White D P, Butler J P, Owens R L, Malhotra A, Wellman A. Acetazolamide improves loop gain but not the other physiological traits causing obstructive sleep apnea. J Physiol 2012; 590:1199-1211.
31. Wellman A, Malhotra A, Jordan A S, Stevenson K E, Gautam S, White D P. Effect of oxygen in obstructive sleep apnea: Role of loop gain. Respir Physiol Neurobiol 2008; 162:144-151.
32. Lai Y Y, Kodama T, Siegel J M. Changes in monoamine release in the ventral horn and hypoglossal nucleus linked to pontine inhibition of muscle tone: An in vivo microdialysis study. J Neurosci 2001; 21:7384-7391.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject having a condition associated with pharyngeal airway collapse, the method comprising administering to a subject in need thereof an effective amount of (i) 4-hydroxyatomoxetine; and (ii) a muscarinic receptor antagonist.

2. The method of claim 1, wherein the muscarinic receptor antagonist comprises oxybutynin.

3. The method of claim 2, wherein the oxybutynin is a substantially pure (R)-oxybutynin.

4. The method of claim 2, wherein the oxybutynin is a racemic mixture of (R)-oxybutynin and (S)-oxybutynin.

5. The method of claim 4, wherein an enantiomeric excess of (R)-oxybutynin relative to (S)-oxybutynin is greater than about 25%.

6. The method of claim 4, wherein an enantiomeric excess of (R)-oxybutynin relative to (S)-oxybutynin is greater than about 50%.

7. The method of any one of claims 2-6, wherein the oxybutynin is in an immediate release formulation.

8. The method of any one of claims 2-6, wherein the oxybutynin is in an extended release formulation.

9. The method of any one of claims 2-8, wherein the oxybutynin is administered at a dosage of from about 2 to about 15 mg.

10. The method of any one of claims 2-6, wherein the oxybutynin is in an immediate release formulation and is administered at a dosage of from about 2.5 to about 10 mg.

11. The method of any one of claims 2-6, wherein the oxybutynin is in an extended release formulation and is administered at a dosage of from about 5 to about 15 mg.

12. The method of any one of claims 1-11, wherein the method further comprises administering to the subject in need thereof an effective amount of a norepinephrine selective reuptake inhibitor (NSRI).

13. The method of claim 12, wherein the NSRI is selected from the group consisting of Amedalin, Atomoxetine, CP-39,332, Daledalin, Edivoxetine, Esreboxetine, Lortalamine, Nisoxetine, Reboxetine, Talopram, Talsupram, Tandamine, Viloxazine, and combinations thereof.

14. The method of any one of claims 1-13, wherein the method further comprises administering to the subject in need thereof an effective amount of a norepinephrine non-selective reuptake inhibitor (NNRI) selected from the group consisting of Amitriptiline, Amoxapine, Bupropion, Ciclazindol, Desipramine, Desvenlafaxine, Dexmethilphenidate, Diethylpropion, Doxepin, Duloxetine, Imipramine, Levomilnacipran, Manifaxine, Maprotiline, Methylphenidate, Milnacipran, Nefazodone, Nortriptyline, Phendimetrazine, Protryptyline, Radafaxine, Tapentadol, Teniloxazine, Venlafaxine, and combinations thereof.

15. The method of any one of claims 1-14, wherein the method further comprises administering to the subject in need thereof an effective amount of Reboxetine.

16. The method of any one of claims 1-15, wherein the 4-hydroxyatomoxetine is administered at a dosage of from about 20 to about 100 mg.

17. The method of any one of claims 1-16, wherein the 4-hydroxyatomoxetine is administered at a dosage of from about 25 to about 75 mg.

18. The method of any one of claims 1-17, wherein the method further comprises:
administering to the subject in need thereof an effective amount of a hypnotic selected from the group consisting of zolpidem, zopiclone, eszopiclone, trazodone, zaleplon, benzodiazepines, gabapentin, tiagabine, and xyrem.

19. The method of any one of claims 1-18, wherein the condition associated with pharyngeal airway collapse is sleep apnea or simple snoring.

20. The method of claim 19, wherein the condition associated with pharyngeal airway collapse is Obstructive Sleep Apnea (OSA).

21. The method of any one of claims 1-20, wherein the subject is in a non-fully conscious state.

22. The method of claim 21, wherein the non-fully conscious state is sleep.

23. The method of any one of claims 1-22, wherein the 4-hydroxyatomoxetine and muscarinic receptor antagonist are administered in a single composition.

24. The method of claim 23, wherein the single composition is an oral administration form.

25. The method of claim 24, wherein the oral administration form is a syrup, pill, tablet, troche, capsule, or patch.

26. A pharmaceutical composition comprising:
4-hydroxyatomoxetine; and
a muscarinic receptor antagonist in a pharmaceutically acceptable carrier.

27. The composition of claim 26, wherein the muscarinic receptor antagonist comprises oxybutynin.

28. The composition of claim 27, wherein the oxybutynin is a substantially pure (R)-oxybutynin.

29. The composition of claim 27, wherein the oxybutynin is a racemic mixture of (R)-oxybutynin and (S)-oxybutynin.

30. The composition of claim 29, wherein an enantiomeric excess of (R)-oxybutynin relative to (S)-oxybutynin is greater than about 25%.

31. The composition of claim 29, wherein an enantiomeric excess of (R)-oxybutynin relative to (S)-oxybutynin is greater than about 50%.

32. The composition of any one of claims 27-31, wherein the oxybutynin is in an immediate release formulation.

33. The composition of any one of claims 27-31, wherein the oxybutynin is in an extended release formulation.

34. The composition of any one of claims 27-33, wherein the oxybutynin is present in an amount of from about 2 to about 15 mg.

35. The composition of any one of claims 27-31, wherein the oxybutynin is in an immediate release formulation and is present in an amount of from about 2.5 to about 10 mg.

36. The composition of any one of claims 27-31, wherein the oxybutynin is in an extended release formulation and is present in an amount of from about 5 to about 15 mg.

37. The composition of any one of claims 26-36, wherein the 4-hydroxyatomoxetine and muscarinic receptor antagonist are disposed together in a pharmaceutically acceptable carrier to form a single dosage agent and/or the 4-hydroxyatomoxetine and muscarinic receptor antagonist are separately disposed in pharmaceutically acceptable carriers to form a separate 4-hydroxyatomoxetine dosing agent and a muscarinic receptor antagonist dosing agent.

38. The composition of any one of claims 26-37, wherein the pharmaceutical composition further comprises a norepinephrine selective reuptake inhibitor (NSRI) selected from the group consisting of Amedalin, Atomoxetine, CP-39,332, Daledalin, Edivoxetine, Esreboxetine, Lortalamine, Nisoxetine, Reboxetine, Talopram, Talsupram, Tandamine, Viloxazine, and combinations thereof.

39. The composition of any one of claims 26-38, wherein the pharmaceutical composition further comprises a norepinephrine non-selective reuptake inhibitor (NNRI) selected from the group consisting of Amitriptiline, Amoxapine, Bupropion, Ciclazindol, Desipramine, Desvenlafaxine, Dexmethilphenidate, Diethylpropion, Doxepin, Duloxetine, Imipramine, Levomilnacipran, Manifaxine, Maprotiline, Methylphenidate, Milnacipran, Nefazodone, Nortriptyline, Phendimetrazine, Protryptyline, Radafaxine, Tapentadol, Teniloxazine, Venlafaxine, and combinations thereof.

40. The composition of any one of claims 26-39, wherein the 4-hydroxyatomoxetine is present in an amount of from about 20 to about 100 mg.

41. The composition of any one of claims 26-40, wherein the 4-hydroxyatomoxetine is present in an amount of from about 25 to about 75 mg.

42. The composition of any one of claims 26-41, wherein the pharmaceutical composition further comprises Reboxetine.

43. The composition of any one of claims 26-42, wherein the pharmaceutical composition further comprises a hypnotic selected from the group consisting of zolpidem, zopiclone, eszopiclone, trazodone, zaleplon, benzodiazepines, gabapentin, tiagabine, and xyrem.

44. The composition of any one of claims 26-43, for use in treating a subject having a condition associated with pharyngeal airway collapse.

45. The composition for use of claim 44, wherein the condition associated with pharyngeal airway collapse is sleep apnea or simple snoring.

46. The composition for use of claim 45, wherein the condition associated with pharyngeal airway collapse is Obstructive Sleep Apnea (OSA).

47. The composition for use of any one of claims 44-46, wherein the subject is in a non-fully conscious state.

48. The composition for use of claim 47, wherein the non-fully conscious state is sleep.

49. A norepinephrine reuptake inhibitor (NRI) comprising 4-hydroxyatomoxetine and a muscarinic receptor antagonist for use in treating a subject having a condition associated with pharyngeal airway collapse.

50. A kit comprising 4-hydroxyatomoxetine and a muscarinic receptor antagonist.

51. The kit of claim 50, for use in treating a subject having a condition associated with pharyngeal airway collapse.

52. A method of treating a subject having a condition associated with pharyngeal airway collapse, the method comprising:
administering to a subject in need thereof an effective amount of a muscarinic receptor agonist; and
modulating a norepinephrine transporter (NET) of the subject in vivo with an effective amount of 4-hydroxyatomoxetine.

53. The method of claim 52, wherein the muscarinic receptor antagonist comprises oxybutynin.

54. The method of claim 53, wherein the oxybutynin is a substantially pure (R)-oxybutynin.

55. The method of claim 53, wherein the oxybutynin is a racemic mixture of (R)-oxybutynin and (S)-oxybutynin.
